(19) United States
(12) Patent Application Publication
(10) Pub. No.: US 2026/0355018 A1
(43) Pub. Date: Nov. 12, 2026

(54) ASSEMBLY COMPRISING AT LEAST ONE DRESSING AND A DEVICE FOR GRIPPING SAID DRESSING BY SUCTION, BLOOD SAMPLING MACHINE AND METHOD

(71) Applicant: BHEALTHCARE, SAINT-HERBLAIN (FR)

(72) Inventors: Sylvain GEFFROY, SAINT-HERBLAIN (FR); Corentin RABOT, SAINT-HERBLAIN (FR); Benjamin HANNES, SAINT-HERBLAIN (FR); Jean-Baptiste DE CHAISEMARTIN, SAINT-HERBLAIN (FR); Aliaume BRETEAU, SAINT-HERBLAIN (FR)

(73) Assignee: BHEALTHCARE, SAINT-HERBLAIN (FR)

(21) Appl. No.: 19/477,103

(22) PCT Filed: Apr. 19, 2024
(86) PCT No.: PCT/EP2024/060835

§ 371 Date: Oct. 20, 2025

(30) Foreign Application Priority Data
Apr. 20, 2023 (FR) FR2304002

(51) Int. Cl. A61F 13/0206; A61B 5/15

(52) U.S. Cl. CPC A61F 13/0206; A61B 5/150053

(57) ABSTRACT

The invention relates to an assembly comprising at least one strip (10) on which are distributed a plurality of dressings (1), a cassette (40), inside which said strip (10) is disposed, and at least one device for gripping said dressing by suction, said dressing comprising:
at least one layer, referred to as absorbent layer (12), comprising at least one absorbent material selected from the group formed from non-woven absorbent materials,
at least one support layer (16) comprising at least one adhesive material, in particular selected from the group formed by polyacrylics, polysiloxanes, copolymers thereof and mixtures thereof, said support layer extending over a surface area greater than the surface area of said absorbent layer.

The invention also relates to an automatic or semi-automatic blood sampling machine comprising such an assembly.

### TECHNICAL FIELD OF THE INVENTION

The invention relates to an assembly comprising at least one dressing and at least one device for gripping said dressing by suction such as a gripping device fitted to an automatic or semi-automatic blood sampling machine. A dressing of such an assembly is suitable for being handled and placed by such a gripping device. The invention also relates to an automatic or semi-automatic blood sampling machine, which makes it possible to place such a dressing on the skin of the patient automatically following blood sampling.

### TECHNOLOGICAL BACKGROUND

Health establishments carry out a very large number of blood collections each day. These operations are time-consuming, repetitive and potentially dangerous for the health care personnel and the patient, given the injury risks associated therewith, linked, for example, to the health care personnel having a tremor, being tired, being inexperienced, the patient moving or reacting in an unhelpful manner when the needle is being introduced into his/her vein.

Furthermore, some patients have veins which are not conducive to the effective collection of blood, which may require several attempts at puncturing by the health care personnel before being able to reach a vein permitting the collection of blood. These repeated attempts may be painful for the patient and can cause injuries, which only complicates the sampling operation for these patients.

Moreover, the worldwide coronavirus health crisis requires that systems be developed which limit contacts between the patients and the health care personnel and/or which can ensure large-scale screening of the population.

Thus, the applicant proposed, in document WO 2021/260035, a blood puncture head and a machine for automatic puncturing into a vein of a limb of a patient comprising a mechatronic assembly, a control unit, a system for taking and processing images of the patient's limb configured to determine an optimal puncture zone.

Such a device thus enables blood sampling operations to be automated.

The puncture head of WO 2021/260035 further comprises a motorized dressing placement arrangement comprising a sucker plate for holding a dressing carried by a hollow shaft pivoting with respect to a chassis between a position for loading a dressing, in which said sucker can come into contact with a dressing dispenser outside said head in order to be able to suck up a dressing by placing said hollow shaft under a vacuum, to a position for placing the dressing, in which said dressing, carried by said sucker, comes into contact pressing against the skin of said limb of the patent at the puncture zone during withdrawal of said needle from said limb of the patient in order to be able to attach said dressing to said limb of the patient by placing said hollow shaft under atmospheric pressure, thus releasing said dressing.

This makes it possible to attach a dressing automatically at the puncture zone while producing a compression point making it possible to avoid any risk of internal or external bleeding. This dressing placement arrangement comprises a sucker which is configured to be able to suck up a dressing, by placing a hollow tube bearing the sucker under vacuum. The sucker comprises, for example, a plurality of orifices arranged on its upper surface in fluid communication with the hollow tube, so that placing the hollow tube under vacuum makes it possible, by pressure difference, to suck up a dressing placed in contact with the upper surface of the sucker, by moving the sucker into said loading position. The dressing dispenser comprises, for example, a roll comprising said dressings spaced apart from one another by a predetermined distance. The sucker comes into contact with a dressing, sucks up the dressing, and the dressing placement arrangement is ready to be used.

This being the case, the currently known dressings do not make it possible to achieve satisfactory conditions for gripping by such a machine, nor dressing placement which is reliable and safe for the patient.

The applicant has thus sought to provide an assembly comprising a gripping device and a dressing particularly suited for being able to be handled by such a gripping device, such as a device of an automatic or semi-automatic blood sampling machine, and for being able to be placed by said gripping device.

Throughout the text, automatic or semi-automatic machine will be understood to be a machine which can operate without human intervention, except for possible operations for loading of the machine, starting up the machine and/or controlling the machine.

### AIMS OF THE INVENTION

The invention aims to provide an assembly comprising at least one dressing and at least one device for gripping said dressing intended to be fitted to an automatic or semi-automatic blood sampling machine.

The invention aims in particular to provide an assembly comprising at least one dressing suitable for being able to be handled and placed by a gripping device such as a suction device of an automatic or semi-automatic blood sampling machine.

The invention also aims to provide, in at least one embodiment of the invention, an automatic or semi-automatic blood sampling machine, fitted with such an assembly which makes it possible to place such a dressing on the skin of the patient automatically following blood sampling.

### DESCRIPTION OF THE INVENTION

In order to do this, the invention relates to an assembly comprising at least one dressing and at least one device for gripping said dressing by suction, said dressing comprising:
at least one layer, referred to as absorbent layer, comprising at least one absorbent material selected from the group formed from non-woven absorbent materials,
at least one support layer comprising at least one adhesive material, said support layer extending over a surface area greater than the surface area of said absorbent layer.

The invention relates more particularly to an assembly comprising at least one strip on which are distributed a plurality of dressings, a cassette, inside which said strip is disposed, and at least one device for gripping said at least one dressing by suction, each dressing comprising:
at least one layer, referred to as absorbent layer, comprising at least one absorbent material selected from the group formed from non-woven absorbent materials,
at least one support layer comprising at least one adhesive material, said support layer extending over a surface area greater than the surface area of said absorbent layer.

The invention thus also relates to such a dressing comprising:
at least one layer, referred to as absorbent layer, comprising at least one absorbent material selected from the group formed from non-woven absorbent materials,
at least one support layer comprising at least one adhesive material, in particular an adhesive material selected from the group formed by polyacrylics, polysiloxanes, copolymers thereof and mixtures thereof, said support layer extending over a surface area greater than the surface area of said absorbent layer.

A dressing in accordance with the invention is adapted to be able to be used by an automatic or semi-automatic blood sampling machine and to make possible easy and automated detachment of each dressing without the need for intervention by an operator and without causing deformation of said dressing liable to hamper the placement of the dressing on the skin of a patient by said suction-gripping device.

Advantageously and in accordance with the invention, each dressing comprises at least one border portion, referred to as detachment border, within which said support layer does not extend beyond said absorbent layer.

In particular, in an advantageous manner and in accordance with the invention, said detachment border portion of each dressing has a contour in the form of an arc of a circle. The inventors have observed that such a curved shape constitutes a starting point for particularly effective detachment of the dressing (with respect to the peelable protector or any other surface on which it is disposed) and facilitates same by the gripping device.

Said support layer can have any geometric shape, for example one selected from polygons or even closed curves. In particular, the general shape of the support layer is considered when it is disposed on a planar support. In particular, in an advantageous manner and in accordance with the invention, said support layer has a peripheral contour extending as a simple closed curve (for example, an ellipse, a circle . . . ) , in particular a substantially circular (or oblong) peripheral contour.

Advantageously and in accordance with the invention, said adhesive material is selected from the group formed by polyacrylics, polysiloxanes, copolymers thereof and mixtures thereof. Each polyacrylic material of said support layer is in particular selected from the group formed by thermoplastic polyacrylics, elastomeric polyacrylics, elastomeric thermoplastic polyacrylics, copolymers thereof and mixtures thereof. Furthermore, said polyacrylics are selected from the group formed by polyacrylates, polymethyl methacrylates, copolymers of butyl acrylate/ethylene, copolymers of methyl acrylate/ethylene, copolymers of methacrylic acid/ethylene, copolymers thereof and mixtures thereof.

With respect to the polysiloxanes, (polymerized silicones or siloxanes) of said support layer, they may be, for example, silicone elastomers.

Advantageously and in accordance with the invention, said support layer of each dressing comprises at least one substrate comprising at least one material selected from the group formed by polyethylenes, copolymers thereof and mixtures thereof. Advantageously and in accordance with the invention, said support layer comprises at least one substrate formed from polyethylene, said adhesive material being distributed over at least a surface portion of said substrate.

Advantageously and in accordance with the invention, said absorbent material of the absorbent layer of each dressing comprises absorbent fibers selected from the group formed by rayon, viscose, cellulosic materials (for example, cotton) and mixtures thereof.

The absorbent layer of each dressing used can be of a different grammage depending on the nature of the materials, the thickness and dimensions and shapes of each layer of said dressing. In particular, in an advantageous manner and in accordance with the invention, said absorbent layer of each dressing has a grammage at least equal to 100 g/m², in particular at least equal to 200 g/m².

Similarly, advantageously and in accordance with the invention, said absorbent layer of each dressing has a thickness between 0.80 mm and 2.50 mm.

Such a thickness and such a grammage contribute to providing a dressing having a fluid-absorbing capacity sufficiently great to permit it to hold, without risk of leakage, all the blood likely to result after the withdrawal of a blood sampling needle from a puncture zone of a patient.

The dressing comprises at least one layer rendering it substantially impermeable to fluids, in particular to air, and to water vapor, in order to facilitate it being gripped by a system of the sucker or suction type. Advantageously and in accordance with the invention, said support layer has a permeability to water vapor which is substantially nil (non-breathable dressing). Advantageously and in accordance with the invention, said support layer of said dressing has a level of transmission of water vapor of less than 1 000 g/m²/24 hours (for example, measured according to standard NF EN 13726-2 of September 2002), and in particular a level of transmission of water vapor which is substantially nil.

Advantageously and in accordance with the invention, said support layer has a permeability to fluids in the liquid state of substantially nil. Similarly, advantageously and in accordance with the invention, said support layer has a permeability to air of substantially nil.

Advantageously and in accordance with the invention, each dressing further comprises a peelable protector. The peelable protector is in the form of a film disposed in contact with the adhesive face of each dressing and is intended to be removed before the dressing is placed on the skin of a patient. Advantageously and in accordance with the invention, each dressing further comprises a peelable protector extending in the form of a strip on which are distributed a plurality of dressings, said absorbent layer being disposed between said support layer and said peelable protector. Such a strip on which are distributed a plurality of dressings can be stored flat, cut out according to the length or number of dressings desired or even be wound on itself or on a support (such as a cylinder, a tube or even a bar). This strip can also be wound directly on an element of said cassette. The assembly formed by the strip of peelable protector on which such dressings are previously disposed thus forms a roll of dressings. There is also nothing to prevent the dressings used in an assembly in accordance with the invention being individualized from each other (unitary dressings), the dressings being, for example, stored stacked one on top of another or side by side placed on their edge (thickness) in a dressing dispenser.

Said strip extends principally along a longitudinal axis on which a plurality of dressings in accordance with the invention are distributed. Advantageously and in accordance with the invention, all the dressings of a single strip are disposed on a single main face of said strip.

The absorbent layer can be of any shape and be disposed in various ways relative to said support layer. In particular, advantageously and in accordance with the invention, said dressings are configured so that each absorbent layer of a dressing extends longitudinally in a direction substantially identical or parallel to said longitudinal axis along which said strip extends. There is also nothing to prevent each absorbent layer of a dressing being made to extend transversely to said longitudinal axis along which said strip extends.

Advantageously and in accordance with the invention, said strip has, on each side of each dressing of said strip, a plurality of holes substantially aligned in a direction parallel to said longitudinal axis. Such holes can serve as markers when the strip is being handled by an automatic or semi-automatic blood sampling machine.

Said adhesive material and/or said absorbent layer can also comprise at least one active substance intended to promote healing or treatment of the skin or of a wound. Such an active substance can, for example, be selected from agents which promote healing such as retinol, vitamin A, vitamin E, antibacterial agents, antiseptics such as chlorhexidine, pain relievers such as paracetamol, local anesthetics or even anti-inflammator.

Advantageously and in accordance with the invention, said cassette comprises a first shell and a second shell which are configured to be able to be fixedly connected to one another so as to form an internal housing for receiving the strip bearing said dressings. Thus, advantageously and in accordance with the invention, said cassette comprises at least one housing for receiving said strip, configured to permit unwinding of said strip.

The two shells can, for example, fit together at least partially one inside the other. The first shell and the second shell may or may not be substantially identical. Each shell of the cassette can comprise stops, notches, clips and/or pins making it possible to fasten them together in a reversible manner. The cassette, and in particular the first shell, has a main portion having an annular central part forming a circular border protruding from a planar bottom. Each shell of the cassette has a substantially planar bottom and peripheral lateral walls. The cassette comprises a guide permitting the strip previously disposed on the annular central part of the cassette to pass through, said guide having a concave border portion. The cassette further comprises a hub configured to be able to be driven in rotation by a motor enabling control of the rotation of said hub to which one end of said strip can be fixed. Such a configuration enables reliable unwinding of the strip via said guide and said previously disposed strip wound on the annular central part of the cassette. The concave border portion has a protruding edge enabling easy and spontaneous detachment of each dressing from the strip, one by one.

Thus, advantageously and in accordance with the invention, said cassette comprises a main portion having an annular central part forming a circular border protruding from a planar bottom, at least one hub configured to be able to be driven in rotation by a motor and a concave border portion having a protruding edge. Thus, said strip can be wound directly on said annular central part of said main portion of said cassette, said annular central part forming a support, around which said strip is wound. There is also nothing to prevent said strip, on which a plurality of dressings are distributed, from being wound on a support (such as a portion of tubing) configured to be able to be positioned on said annular central part of said main portion of said cassette. Such a presentation of said strip at least partially wound on itself has the advantage of facilitating its handling, its storage and its loading within a cassette.

Such a cassette has the advantage of being able to be in the form of a part which is inexpensive while being robust, safe, reliable and reusable (not necessarily disposable or for single use only). In fact, a strip of the desired length and having a desired number of dressings can be adapted to each case. Such a cassette makes it possible in particular to receive a large number of dressings (for example, up to 1 000 dressings) so as to limit the amount of handling required in relation to reloading.

The assembly in accordance with the invention can also comprise at least one sensor for detecting the presence of a dressing facing said gripping device. Other types of sensors can also be provided in order to detect the position or possible jamming of a dressing in the zone where the dressings are detached from the strip corresponding to said protruding edge of the cassette.

Advantageously and in accordance with the invention, the suction-gripping device is connected to an automated arm such as an arm comprising a hollow tube subject to a pressure enabling at least one dressing from said strip to be gripped by suction.

Said suction-gripping device can be of any form enabling said dressing to be handled by suction. Advantageously and in accordance with the invention, said suction-gripping device comprises a suction plate having at least one suction hole connected to a source of suction. Such a suction plate makes it possible to apply a pressure strictly lower than atmospheric pressure, in particular a pressure between 40 MPa and 60 MPa, within, for example, a hollow tube in fluid communication with each suction hole of the suction plate. Said suction plate comprises a predetermined number of holes, said holes having a predetermined diameter, adapted to enable the gripping of a dressing in accordance with the invention.

The invention also relates to a machine for automatic or semi-automatic sampling of blood from a limb of a patient, comprising at least one assembly in accordance with the invention.

Such a blood sampling machine can comprise, for example, a mechatronic assembly, a unit for controlling said mechatronic assembly, a system for capturing and processing images of the patient's limb configured to determine an optimal puncture zone, characterized in that it further comprises a puncture head mounted on said mechatronic assembly. Such a machine is described, for example, in the application WO 2015/158978 in the applicant's name.

The invention also relates to a method for using an assembly in accordance with the invention, comprising the following steps:
a cassette is provided, inside which a strip is disposed, on which a plurality of dressings are distributed, said strip being in a form in which it is at least partially wound on itself, disposed in the main portion of the cassette and having a portion passing inside a guide and extending to a hub of the cassette,
the suction-gripping device lifts a first dressing disposed on said strip at the outlet of the guide of the cassette,
after having lifted said dressing, said strip is moved so as to enable the positioning of a second dressing at the outlet of the guide of the cassette.

The invention also relates to an assembly, a machine and a method of using such an assembly, which are characterized in combination by all or some of the features mentioned above or below.

### LIST OF FIGURES

Other aims, features and advantages of the invention will become apparent upon reading the following description given solely in a non-limiting way and which makes reference to the attached figures in which:

FIG. 1 is a schematic perspective view of a dressing in accordance with one embodiment of the invention,

FIG. 2 is a schematic cross-sectional view of a dressing in accordance with the embodiment of FIG. 1,

FIG. 3 is a schematic view from above of a portion of a strip in accordance with one embodiment of the invention,

FIG. 4 is a schematic perspective view of a strip wound in the form of a roll comprising a strip in accordance with one embodiment of the invention,

FIG. 5 is a schematic view of an automatic blood sampling machine in accordance with one embodiment of the invention,

FIG. 6 is a schematic view of an assembly in accordance with one embodiment of the invention,

FIG. 7 is a schematic perspective view of a cassette of an assembly in accordance with one embodiment of the invention.

### DETAILED DESCRIPTION OF AN EMBODIMENT OF THE INVENTION

In the figures, for the purposes of illustration and clarity, scales and proportions have not been strictly respected.

Furthermore, identical, similar or analogous elements are designated by the same reference signs in all the figures.

An assembly in accordance with the invention comprises at least one dressing 1 and at least one device 8 for gripping such a dressing by suction (or vacuum suction).

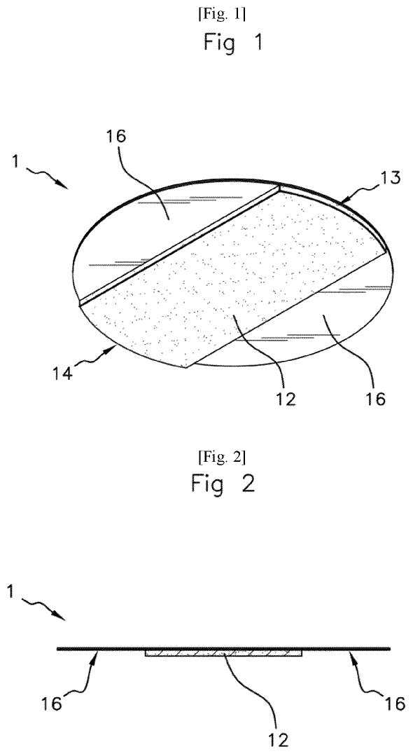

FIG. 1 shows a dressing 1 having a circular peripheral contour comprising an absorbent layer 12 and a support layer 16 which are juxtaposed and superimposed with respect to one other. FIG. 2 shows a transverse cross-section of the dressing 1 illustrated in FIG. 1.

The absorbent layer 12 comprises at least one absorbent material and is in the form of a small strip having two straight lateral edges which are parallel to one another, and two longitudinal ends each forming a curved edge.

The absorbent material is selected from the group formed by non-woven absorbent materials and comprises absorbent fibers such as rayon, viscose, cellulosic materials (for example, cotton) or a mixture of such fibers. The absorbent layer has a grammage at least equal to 100 g/m², in particular at least equal to 200 g/m², for example a grammage between 300 g/m² and 600 g/m². The absorbent layer preferably has a thickness between 0.80 mm and 2.50 mm, in particular a thickness between 1.2 mm and 2.0 mm.

The support layer 16 covers the absorbent layer 12 and extends beyond the lateral edges of the absorbent layer small strip, forming two wings of the dressing. The support layer 16 thus has a surface area greater than the surface area of the absorbent layer 12. The support layer 16 comprises at least one adhesive material making it possible to cause said absorbent layer 12 to adhere thereto and to cause the dressing to be held on the skin of the patient in contact with whom it will be applied. The adhesive material is in particular selected from the group formed by polyacrylics, polysiloxanes, copolymers thereof and mixtures thereof.

The dressing has two border portions 13, 14, within which the support layer 16 does not extend beyond the absorbent layer 12, each border portion 13, 14 having a non-zero curvature in a constant direction, or, in the embodiment illustrated in FIGS. 1 to 4, an arc of a circle.

The dressing and the support layer can also have a peripheral contour extending on a simple closed curve such as a circle, for example an ellipse.

Each polyacrylic material of the support layer is in particular selected from the group formed by thermoplastic polyacrylics, elastomeric polyacrylics, elastomeric thermoplastic polyacrylics, copolymers thereof and mixtures thereof. Furthermore, said polyacrylics are selected from the group formed by polyacrylates, polymethyl methacrylates, copolymers of butyl acrylate/ethylene, copolymers of methyl acrylate/ethylene, copolymers of methacrylic acid/ethylene, copolymers thereof and mixtures thereof.

With respect to the polysiloxanes (polymerized silicones or siloxanes), they may be, for example, silicone elastomers.

According to a first option, said support layer 16 can be formed of a single material (which, as appropriate, can itself be formed from a mixture of materials). According to this first option, the adhesive material is in the form of a thin film having two main faces, one having adhesive properties and being oriented towards the absorbent layer and being intended to be positioned in contact with the skin of a patient, the other being able to form the outer surface of the dressing (and thus non-adhesive but protective).

According to a second option, the support layer 16 can comprise, in addition to the adhesive material, at least one substrate formed at least in part from polyethylene. Thus the adhesive material can be distributed at least partially on the surface of the substrate.

In the case of these two options, the support layer, by reason of the nature of the adhesive material and/or the nature of the substrate, renders the dressing sufficiently impermeable to fluids (liquids or gasses) and to water vapor to facilitate it being gripped by a suction-gripping device 8. The support layer thus has a substantially zero permeability to water vapor (so that the dressing is said to be non-breathable) as well as a substantially zero permeability to fluids in the liquid state.

The support layer has no perforations in its thickness, i.e. it is continuous and has no through-holes. This makes it possible to limit the permeability to fluids, including water vapor, of said dressing in order to make it easier to handle by the suction-gripping device.

The support layer preferably has a thickness between 0.05 mm and 1.00 mm, in particular a thickness between 0.10 mm and 0.50 mm, for example a thickness of the order of 0.20 mm.

The dressing can comprise a peelable protector 20 protecting the adhesive face of the support layer.

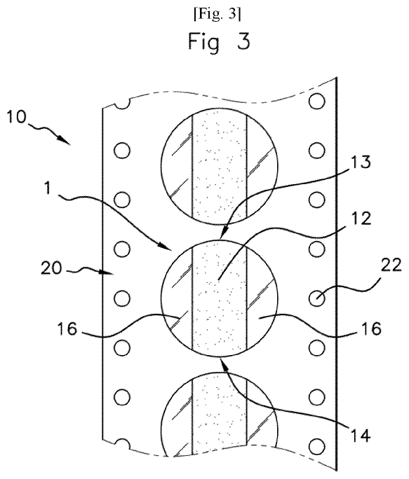

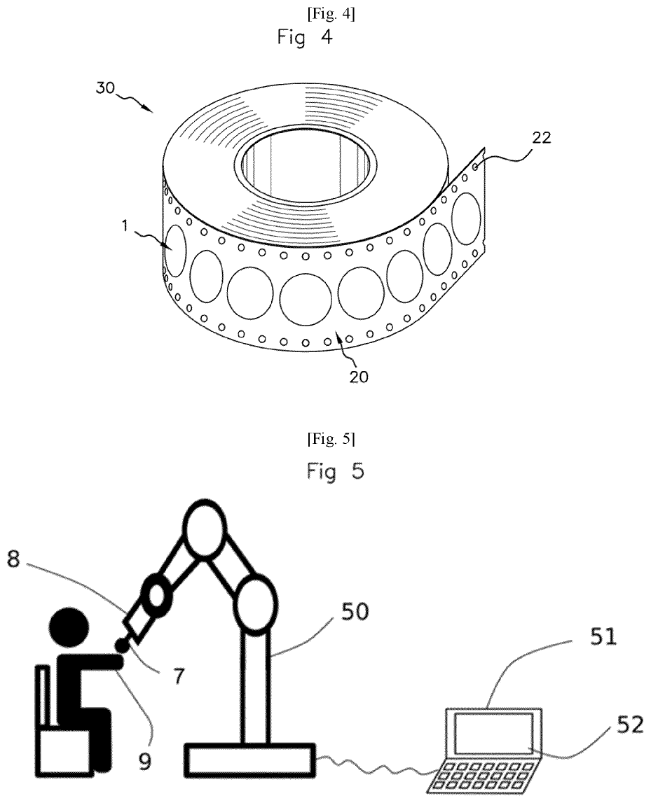

This peelable protector 20 can be used to prepare a strip 10, illustrated in FIG. 3, extending mainly along a longitudinal axis and comprising a plurality of dressings in accordance with the invention. The dressings are, in particular, disposed aligned with respect to each other along the longitudinal axis of the strip 10. The peelable protector 20 is intended to be removed just before the dressing 1 is placed on the skin of the patient. Furthermore, in the embodiment illustrated in FIGS. 3 and 4, the strip 10 further has, on either side of each dressing, a plurality of holes 22 substantially aligned in a direction parallel to the longitudinal axis on which the strip 10 extends. It will be noted that the longitudinal axis of the strip 10 is designated when considering the strip 10 extended or unwound, for example on a planar object. However, the strip can be wound at least in part on itself (on or not on a support) and be in the form of a roll 30 (FIG. 4) in order to store a predetermined number of dressings while limiting the volume it takes up, such a roll 30 being able to be fitted to an automatic blood sampling machine.

In the embodiment illustrated in FIGS. 1 to 7, and as can be seen in FIGS. 3 and 4, the absorbent material of the absorbent layer of each dressing of a roll is configured to extend principally in a single longitudinal direction. Thus each small strip of absorbent material of a dressing is aligned with the small strip of absorbent material of the following (or preceding) dressing. The dressings are positioned on a strip so that each absorbent layer of a dressing extends longitudinally in a direction substantially identical or parallel to the longitudinal axis along which said strip extends (when it is disposed on a planar support and not when it is wound on itself).

Said adhesive material and/or said absorbent layer can also comprise at least one active substance intended to promote healing or treatment of the skin or of a wound. Such an active substance can, for example, be selected from agents which promote healing such as retinol, vitamin A, vitamin E, antibacterial agents, antiseptics such as chlorhexidine, pain relievers such as paracetamol, local anaesthetics or even anti-inflammatories.

According to one exemplified embodiment such as that illustrated in FIGS. 1 and 2, a dressing is prepared from a support layer formed of polyethylene, being in the form of a disc with a diameter of 30 mm and covered over its whole surface (on a single main face) with an acrylic adhesive. This support layer has a substantially constant thickness of the order of 0.2 mm. A strip of absorbent material is cut having a width of 14 mm formed of a mixture of viscose and fibers of polyethylene (PE) and of polyethylene terephthalate (PET). The absorbent strip has a thickness of 1.5 mm and a grammage of 100 g/m². The strip of absorbent material is applied to the center of said adhesive support layer and in contact with the adhesive face thereof. The dressing may then possibly be covered by applying a peelable protective film on the side where the absorbent material is and so as to protect the adhesive surface portions of the dressing.

The suction-gripping device 8 comprises, for example, a suction plate having at least one suction hole connected to a source of suction configured to make it possible to reach a pressure strictly lower than atmospheric pressure within, for example, a hollow tube in fluid communication with each suction hole of the suction plate. It will suffice, in particular, to reach a suction pressure between 40 MPa and 60 MPa in order to permit gripping and handling of such dressings.

The invention also relates to an automatic blood sampling machine 50 illustrated very schematically in FIG. 5. This machine comprises, in the illustrated embodiment, a device 8 for gripping a dressing 7, a unit 51 for controlling the gripping device 8, a system 52 for capturing and processing images of the patient's limb, configured to determine an optimal puncture zone where the dressing 7 must also be placed, and a puncture head (not shown). The system 52 for capturing and processing images comprises, for example, a camera, not illustrated in the figures, adapted to acquire the limb of the patient 9 to be punctured. The images are then transmitted to an image processing module configured to detect an optimal vein for puncturing. The method of identification of the optimal vein is, for example, that described in the application WO2015158978 in the applicant's name. Once the optimal vein has been detected, the control unit 51 transmits, to the actuators of the gripping device 8 and to the actuators of the puncture head, the movement information for the needle holder so as to allow insertion of a needle into the limb of the patient 9 from which blood is to be sampled.

Prior to the blood sampling operation, the puncture head in accordance with the invention is loaded with sampling tubes required for the specific analysis of the patient. The tubes are inserted into the rotating barrel, which preferably comprises a number of housings for receiving tubes, greater than or equal to the number of tubes necessary for the examination effected on the patient.

The suction-gripping device 8 then lifts a dressing 7 from a roll of dressings fitted in the sampling machine and the dressing 7 can be placed on the skin of the patient 9 where the blood sampling has been carried out.

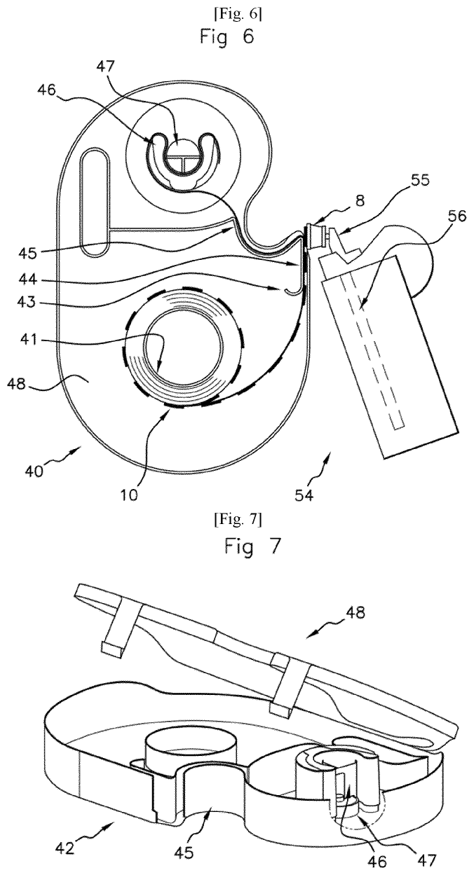

FIG. 6 illustrates more precisely an assembly in accordance with the invention comprising a suction-gripping device 8 and a plurality of dressings as well as a cassette 40, within which the strip 10 comprising the dressings is disposed. FIG. 7 illustrates an open cassette 40, the cassette 40 comprising two shells 42, 48 which can be fitted one within the other. Each shell 42, 48 of the cassette can comprise stops, notches, clips and/or pins making it possible to fit them one within the other so as to fixedly attach (preferably reversibly) the first shell 42 and the second shell 48, providing an internal housing for receiving the strip 10 bearing dressings. The cassette 40, and in particular the first shell 42, has a main portion having an annular central part 41 forming a circular border protruding from a planar bottom of the first shell 42 and making it possible to receive the strip 10 comprising the dressings at least partially wound on itself. The strip 10 can be wound directly on the annular central part 41 of the cassette 40 which then forms the support around which the strip 10 is wound or around which the support for the strip will be positioned when the strip 10 is wound on a support (the winding support of the strip 10 thus having an internal diameter substantially identical to, or slightly greater than, the diameter or the external dimensions of the annular central part 41 of the cassette 40). The strip 10 can thus be wound on itself on a support such as a portion of tubing configured to be able to be positioned on said annular central part of said main portion of said cassette. Such a wound form of the strip makes it possible to facilitate its handling, its storage and its loading/unloading within a cassette, in particular in order to permit the replacement of a strip 10 when all the dressings on it have been lifted.

It is also possible to make provision for the strip to be folded on itself, dressing by dressing, concertina fashion, instead of being wound on itself. The cassette can be adapted to being disposed in this manner while allowing it to be used in the same way for the lifting of the dressings by the gripping device.

Thus, each shell of the cassette 40 has a substantially planar bottom and peripheral lateral walls. The cassette 40 comprises a guide 44 having a concave border portion 45 enabling passage of the strip 10 unwound from the main portion of the cassette 40, the strip 10 bearing the dressings being previously disposed on the annular central part 41 of the cassette. In the illustrated embodiment, the guide 44 has, on the side where the strip 10 enters, an end 43 curved towards the outside of the passage which it forms.

The cassette 40 further comprises a hub 46 laterally offset with respect to the main portion comprising the annular central part 41, and configured to be able to be driven in rotation by a motor 47 making it possible to control the rotation of the hub 46 to which the end of the strip 10 coming from the guide 44 can be fixed so as to enable the strip 10 to unwind from the annular central part 41 of the cassette. In the illustrated embodiment, the hub has a C-shaped or omega-shaped transverse cross-section around a cylindrical portion. Any other shape is also possible, such as a simple portion of a cylinder or a rod. Such a C shape or omega shape has the advantage of being able to form a passage wedging or pinching the strip, making it possible to hold it in position between the C-shaped (or omega-shaped) portion and a central portion and thus to enable storage of the peelable protector 20 as the strip bearing the dressings is being unwound.

The concave border portion 45 has a protruding edge, facing which the suction plate of the suction-gripping device 8 may approach, in contact with which a dressing can spontaneously become detached from the strip 10 in passing this protruding edge of the concave border portion 45. The suction-gripping device 8 is connected to an automated arm 54, such as an arm of an automatic blood sampling machine 50, by a piece 55, and connected to a hollow tube 56 subject to a pressure enabling the dressing lifted from the strip 10 to be gripped by suction.

The direction of rotation of the strip, on which a plurality of dressings are distributed, for it to unwind within the cassette, and of the hub making it possible to wind the strip without dressings after detachment thereof can be identical or different.

Such an assembly in accordance with the invention is used according to a method having the following steps:
such a cassette is provided, inside which a strip 10 is disposed, on which a plurality of dressings 1 are distributed, said strip being in a form in which it is at least partially wound on itself, disposed in the main portion of the cassette 40 and having a portion passing inside a guide 44 and extending to a hub 46 of the cassette 40,
the suction-gripping device 8 lifts a first dressing disposed on said strip at the outlet of the guide 44 of the cassette 40,
after having lifted said dressing, the strip is moved so as to enable the positioning of a second dressing at the outlet of the guide 44 of the cassette 40.

1. An assembly comprising at least one strip on which are distributed a plurality of dressings, a cassette, inside which said strip is disposed, and at least one device for gripping said at least one dressing by suction, each dressing comprising:
at least one layer, referred to as absorbent layer, comprising at least one absorbent material selected from the group formed from non-woven absorbent materials,
at least one support layer comprising at least one adhesive material, said support layer extending over a surface area greater than the surface area of said absorbent layer.

2. The assembly as claimed in claim 1, wherein each dressing comprises at least one border portion, referred to as detachment border, within which said support layer does not extend beyond said absorbent layer.

3. The assembly as claimed in claim 2, wherein each detachment border portion of each dressing has a contour in the form of an arc of a circle.

4. The assembly as claimed in claim 1 wherein said adhesive material is selected from the group formed by polyacrylics, polysiloxanes, copolymers thereof and mixtures thereof.

5. The assembly as claimed in claim 1, wherein said support layer of each dressing comprises at least one substrate comprising at least one material selected from the group formed by polyethylenes, copolymers thereof and mixtures thereof.

6. The assembly as claimed in claim 1 wherein said absorbent material of each dressing comprises absorbent fibers selected from the group formed by rayon, viscose, cellulosic materials and mixtures thereof.

7. The assembly as claimed in claim 1, wherein said absorbent layer of each dressing has a grammage at least equal to 100 g/m², in particular at least equal to 200 g/m².

8. The assembly as claimed in claim 1 wherein said absorbent layer (12) of each dressing has a thickness between 0.80 mm and 2.50 mm.

9. The assembly as claimed in claim 1 wherein said support layer of each dressing has a peripheral contour extending as a simple closed curve.

10. The assembly as claimed in claim 1 wherein said cassette comprises a first shell and a second shell configured to be able to be fixedly attached to one another.

11. The assembly as claimed in claim 1 wherein said cassette comprises at least one housing for receiving said strip, configured to permit unwinding of said strip.

12. The assembly as claimed in claim 1 wherein said cassette comprises a main portion having an annular central part forming a circular border protruding from a planar bottom, at least one hub configured to be able to be driven in rotation by a motor and a guide having a concave border portion having a protruding edge.

13. The assembly as claimed in claim 1 one further comprising a peelable protector extending in the form of said strip on which are distributed a plurality of dressings, said absorbent layer being disposed between said support layer and said peelable protector.

14. An automatic or semi-automatic blood sampling machine, further comprising at least one assembly comprising at least one strip on which are distributed a plurality of dressings, a cassette, inside which said strip disposed, and at least one device—for gripping said at least one dressing by suction, each dressing comprising:
at least one layer, referred to as absorbent layer, comprising at least one absorbent material selected from the group formed from non-woven absorbent materials,
at least one support layer comprising at least one adhesive material, said support layer extending over a surface area greater than the surface area of said absorbent layer.

15. A method for using at least one assembly as claimed in claim 12 comprising the following steps:
such a cassette is provided, inside which said strip is disposed, on which a plurality of dressings are distributed, said strip being in a form in which it is at least partially wound on itself, disposed in said main portion of the cassette and having a portion passing inside said guide and extending to a hub of the cassette,
the suction-gripping device lifts a first dressing disposed on said strip at the outlet of the guide of the cassette,
after having lifted said dressing, said strip is moved so as to enable the positioning of a second dressing at the outlet of the guide of the cassette.
